(19) United States
(12) Plant Patent Application Publication
(10) Pub. No.: US 2019/0307038 P1
(43) Pub. Date: Oct. 3, 2019

(54) Poplar tree named 'Gold Poplar'

(50) ;

(71) Applicant: Changchun Senfeng Agroforestry Technology Development Co., Ltd, Huadian City (CN)

(72) Inventors: Wang Xingfu, Huadian City (CN); Wang Xiaoduo, Huadian City (CN)

(21) Appl. No.: 16/501,288

(22) Filed: Mar. 19, 2019

(30) Foreign Application Priority Data
Mar. 29, 2018 (CN) 20180185

(51) Int. Cl. A01H 6/00

(52) U.S. Cl. PLT/258

(57) ABSTRACT

A new and distinct poplar tree variety, Populus, cv. ‘Gold poplar’ is characterized by having tender yellow color leaves in the spring (Y100,C10,M5,B0), green and yellow leaves in the summer (Y100,C20,M10,B0) and gold leaves in the fall (Y100,C0,M40,B0) with a red petiole (Y100,M100,C20,B0).

Latin name of the genus claimed: Populus.

Variety denomination: ‘Gold Poplar’.

### REFERENCE TO RELATED APPLICATIONS

The present application claims the benefit under 35 U.S.C. § 119(c) of Chinese PBR Application No. 20180185, filed Mar. 29, 2018, which is incorporated herein by reference in its entirety under 37 C.F.R. § 1.57.

### BACKGROUND AND SUMMARY OF THE INVENTION

This application relates to the discovery and asexual propagation of a new and distinct variety of Populus cv. ‘Gold poplar’. The new variety was found in July 2011 among Xiao Hei Yang trees (Populus X xiaohei) that were planted in Ba dao he zi Village, Huadian, Jili City, Jilin Province, China. The new variety was discovered and asexually propagated by Wang Xingfu and Wang Xiaoduo and after observation to confirm stable and consistent traits was given the name of ‘Gold poplar’ in 2017.

The new variety ‘Gold poplar’ has been shown to maintain its distinguishing characteristics through successive asexual propagations by, for example, cuttings. Spikes of the new variety ‘Gold poplar’ were collected in June 2012, from which more than 100 seedlings were cultivated using the method of twig cuttings. The resulting spikes were collected in December 2012 and in the spring of 2013 more than 800 seedlings were planted. By 2017 more than 80,000 seedlings were bred. Trees of the new variety have moderate growth, strong cold resistance and excellent ornamental properties, with leaves having a golden color in the fall It was observed that the traits were stable and consistent throughout multiple generations and over many years.

The characteristic which distinguishes the new variety from other known poplar varieties is the leaf color. The new variety ‘Gold poplar’ has tender yellow colored leaves in the spring, yellow green colored leaves in the summer and gold colored leaves in the fall, with a red petiole.

The new variety ‘Gold poplar’ differs from Xiao Hei Yang (Populus X xiaohei) and Qing Yang(Populus cathayana) in that the new variety has a tender yellow leaf color in the spring and yellow green leaf color in the summer while the two comparative varieties have green color leaves in both spring and summer. Additionally, the new variety ‘Gold poplar’ has a golden color leaf in the fall, while Xiao Hei Yang (Populus X xiaohei) and Qing Yang (Populus cathayana) have yellow-colored leaves in the fall.

The new variety ‘Gold poplar’ has the same range of day-time growing temperatures as other common outdoor Populus cultivars.

### BRIEF DESCRIPTION OF THE PHOTOGRAPH

The accompanying photographs show typical specimens of the new variety, designated ‘Gold poplar’ in the illustrations, including foliage, trunk and stems, in color as nearly true as it is reasonably possible to make in color illustrations of this character.

The photographs of the new variety ‘Gold poplar’ were taken in China in 2017 and 2018.

FIGS. 1 and 2 show one-year-old trees with two-year-old roots and one-year-old stems of the new variety ‘Gold poplar’ in the summer of 2018 in Huadian, Jilin, China.

FIG. 3 shows three-year-old trees with three-year-old roots and three-year-old stems of the new variety ‘Gold poplar’ in the summer of 2018 in Huadian, Jilin, China.

FIGS. 4, 5 and 6 show one-year-old trees with one-year-old roots and one-year-old stems of the new variety ‘Gold poplar’ in the summer of 2018 in Longtan, Jilin, China.

FIGS. 7 and 8 show one-year-old trees with two-year-old roots and one-year old-stem of the new variety ‘Gold poplar’ in the summer of 2017 in Huadian, Jilin, China.

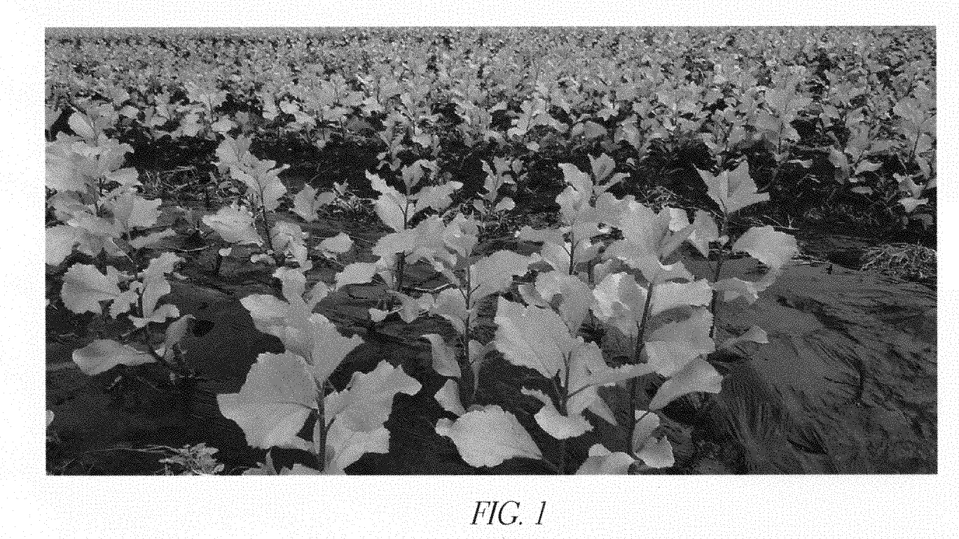

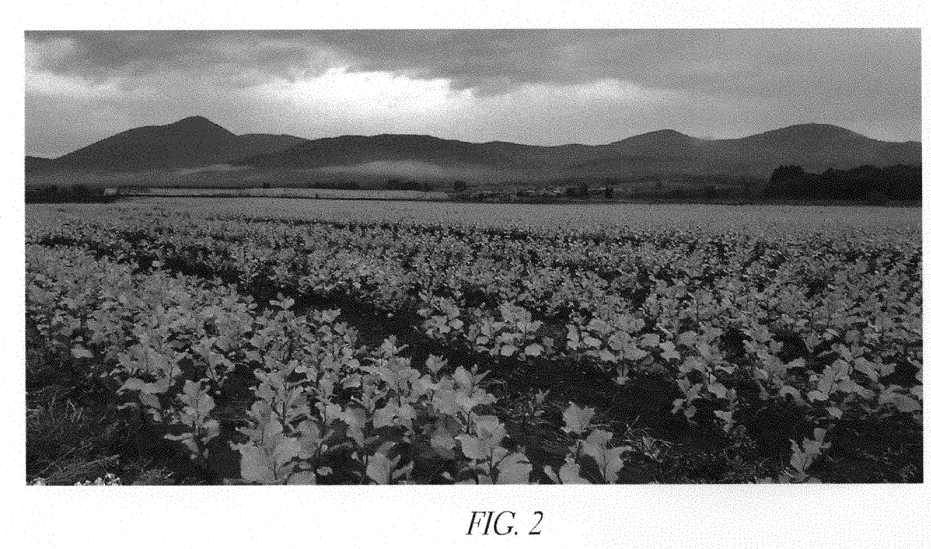

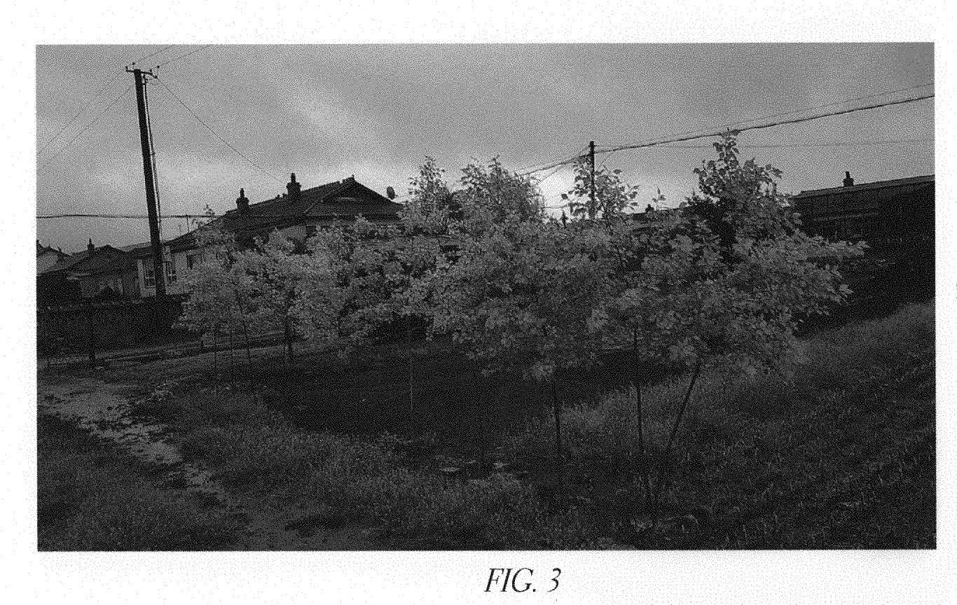

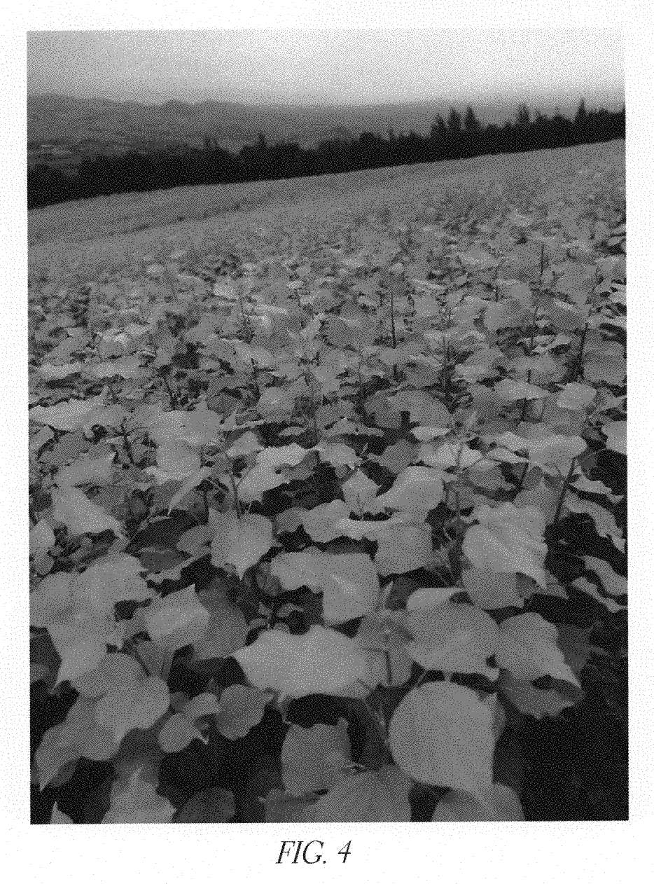

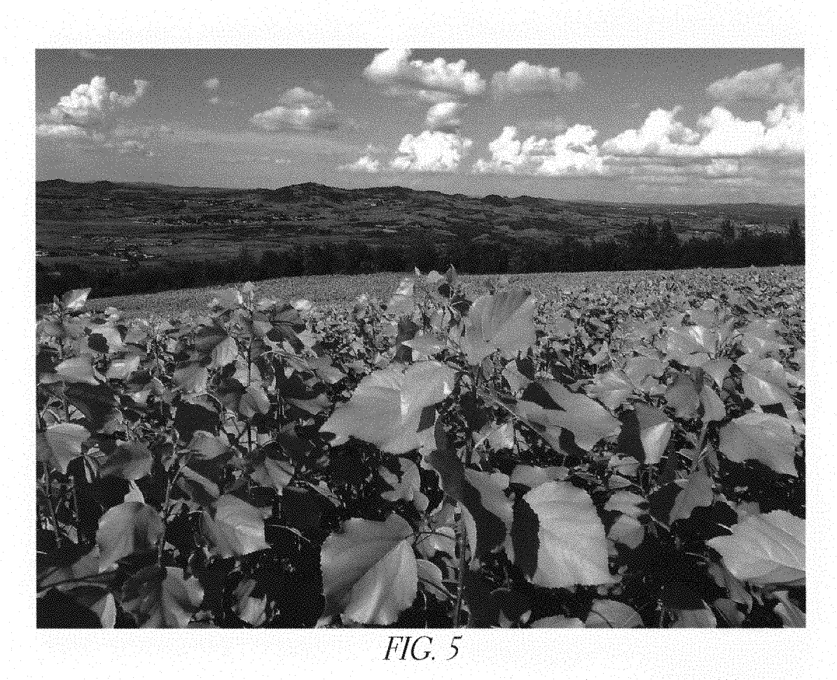

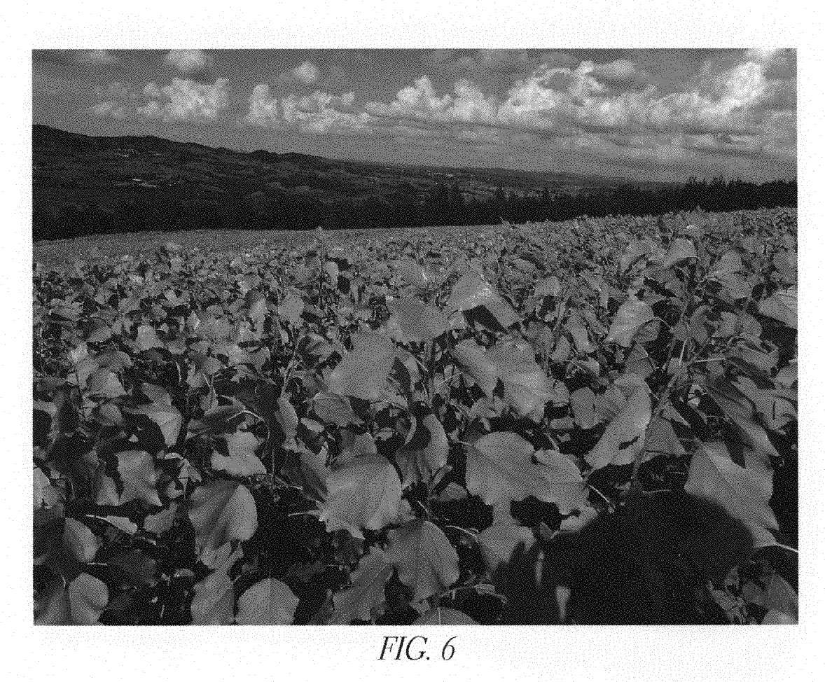

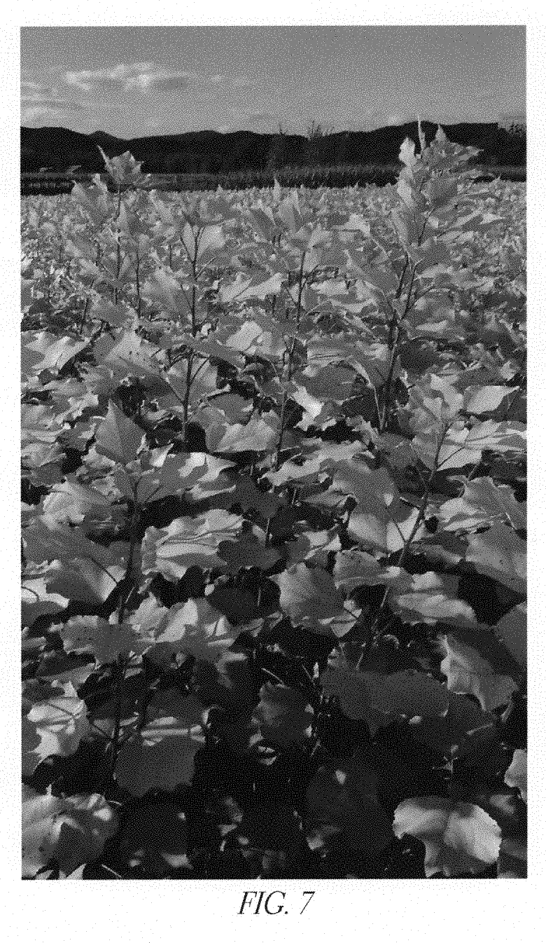

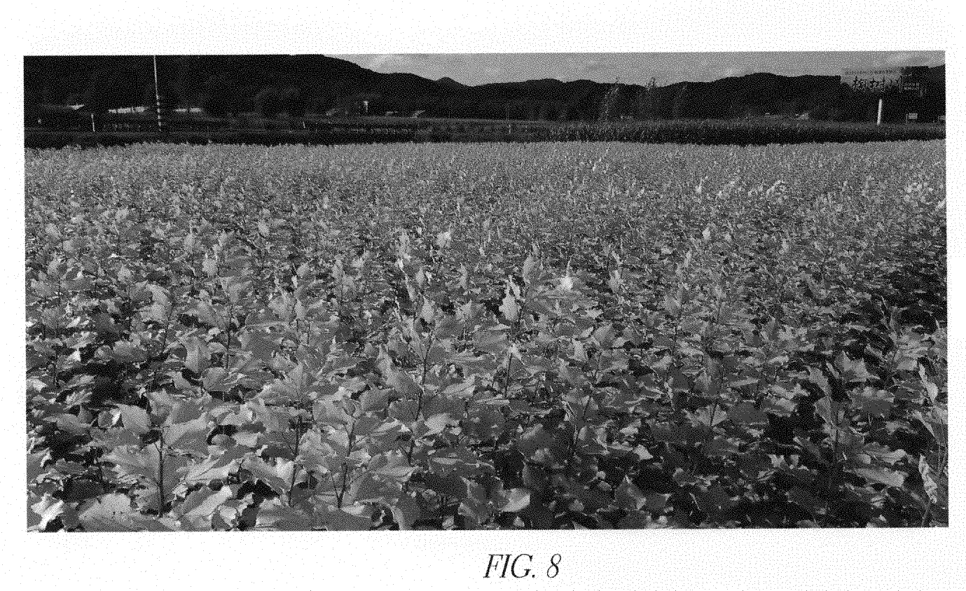

### DESCRIPTION OF THE NEW VARIETY

Throughout this specification, color names beginning with a small letter signify that the name of that color, as used in common speech is aptly descriptive. Color number desciptions were obtained using the Complete Process Colour Finder (Art Distribution Center Ltd., 1996).

The descriptive matter which follows pertains to three-year-old ‘Gold Poplar’ plants grown in Jilin, China and evaluated on Mar. 29, 2018. It is believed to apply to plants of the variety grown under similar conditions of soil and climate elsewhere.
Plant:
   Growth potential.—Weak.
   Shape of trunk.—Straight.
   Shape of crown.—Ovate.
   Bark cracking.—Absent (young tree).
Stem:
   Shape of trunk.—Straight.
   Shape of crown.—Ovate.
   Angle on ¾ height stem.—Present.
Stem:
   Groove depth on ¾ stem.—Medium.
   Color of sunny side on ¾ stem.—About reddish brown.
   Color of nightside on ¾ stem.—About pale red.
   Mucilages on ¾ height stem.—Present.
   Floss on ¾ height stem.—Absent.
   Angle on ½ height stem.—Present.
   Groove depth on ½ stem.—Shallow.
   Color of sunny side on ½ stem.—About light green.
   Color of nightside on ½ stem.—About grey green.
Lenticels:
   Distribution.—Irregular.
   Shape.—Circle.
Lateral branch:
   Quantity.—Large.
   Angle.—Medium.
   Attitude.—Upward.
Leaf:
   Leaf number of glands at base of leaf.—Irregular.
   Leaf posture.—Upward (spring and summer).
Leaf bud:
   Shape.—Long ovate.
   Length.—Medium.
   Color.—About reddish brown.
   Apex shape.—Acuminate.
   Way of growing.—Adhesion.
Leaf blade:
   Color in spring.—About tender yellow (Y100,C10,M5,B0).
   Color in summer.—About yellow-green (Y100,C20,M10,B0).
   Color in autumn.—About golden (Y100,C0,M40,B0).
   Depth of young leaf color.—Medium.
   Length.—Medium.
   Width.—Medium.
   Ratio of midrib length to maximum width of blade.—0.9-1.1.
   Anthocyanin in the middle vein of the upper surface.—Present.
   Distribution of anthocyanin in middle vein of upper surface.—From base to middle.
   Anthocyanin intensity of upper surface midvein.—Medium.
   Angle between midrib and second first lateral vein at lower end.—Approximately 40°-49°.
   Upper surface villus distribution.—Absent.
   Lower surface villus distribution.—Absent.
   Surface profile.—Flat.
   Degree of concavity and convexity between veins.—Weak.
   Base shape.—Broad wedge.
   Shape at the junction with petiole.—Micro concave.
   Type of tip.—Short tail tip.
   Lobes.—Absent.
Petiole:
   Color.—About red (Y100,M100,C20,B0).
   Length.—Medium.
   Ratio of petiole length to main vein length.—0.51-0.60.
   Sectional shape in middle.—Ellipse.
   Floss.—Absent.
   Upper surface color.—About red.
Resistance:
   Insect resistance.—Medium.
   Disease resistance.—Medium resistance to black spot disease, rust disease and canker.

What is claimed is:

1. A new and distinct variety of poplar tree as herein described and illustrated.
